(19) United States
(12) Patent Application Publication
(10) Pub. No.: US 2022/0291239 A1
(43) Pub. Date: Sep. 15, 2022

(54) METHODS FOR DETERMINING THE SEVERITY AND PROGNOSIS OF RSV INFECTION

(71) Applicants: UNIVERSITEIT ANTWERPEN, Antwerpen (BE); UNIVERSITAIR ZIEKENHUIS ANTWERPEN, Edegem (BE)

(72) Inventors: Peter DELPUTTE, Kortrijk (BE); Annemieke SMET, Sint-Niklaas (BE); Stijn VERHULST, Antwerpen (BE); Benedicte DE WINTER, 's-Gravenwezel (BE); Winke VAN DER GUCHT, Wilrijk (BE); Kim STOBBELAAR, Wilrijk (BE)

(21) Appl. No.: 17/761,383

(22) PCT Filed: Sep. 21, 2020
(86) PCT No.: PCT/EP2020/076323

§ 371 Date: Mar. 17, 2022

(30) Foreign Application Priority Data
Sep. 20, 2019 (EP) 19198638.9

(51) Int. Cl. G01N 33/68

(52) U.S. Cl. CPC G01N 33/6893; G01N 2333/135; G01N 2333/4725; G01N 2800/12

(57) ABSTRACT

An in vitro method is provided for determining the severity and/or prognosis of a respiratory syncytial virus (RSV) infection in a subject, based on the expression of one or more mucin genes in respiratory epithelial cells. The respiratory epithelial cells can be isolated from a biological sample of a subject, or they can be cultured in vitro and exposed to a biological sample of the subject.

### CROSS-REFERENCES TO RELATED APPLICATIONS

This application is a national-stage application under 35 U.S.C. § 371 of International Application No. PCT/EP2020/076323, filed Sep. 21, 2020, which International Application claims benefit of priority to European Patent Application No. 19198638.9, filed Sep. 20, 2019.

### TECHNICAL FIELD

The present invention is situated in the field of respiratory syncytial virus (RSV) infections. In particular, the present invention provides an in vitro method for determining the severity and/or prognosis of an RSV infection based on the expression of one or more mucin genes.

### BACKGROUND

Respiratory syncytial virus (RSV), recently renamed to human Orthopneumovirus, is the most important viral respiratory pathogen in infants and children, adult patients with immunodeficiency or cardiopulmonary disease and is recognized as a major threat for the elderly population. An RSV infection starts with typical common-cold like symptoms but may progress to serious lower respiratory tract infections associated with a high rate of hospitalization of infants, children and elderly. No vaccines or therapeutics are available except for Synagis®, also known as the humanized antibody palivizumab. Palivizumab, which is solely used for passive immunization of high-risk infants, targets a specific, highly conserved epitope on the fusion protein, resulting in fusion inhibition. Currently, treatment of severe lower respiratory tract infections as a result of RSV infection consists of supportive care only, such as oxygen administration and nutrition.

RSV is classified in the family of Pneumoviridae, genus Orthopneumovirus and can be divided into two subtypes, RSV-A and RSV-B. It has a non-segmented, negative, single stranded RNA genome that consists of 10 genes, encoding 11 proteins. The viral envelope contains three proteins: the attachment protein (G), the fusion protein (F), and the small hydrophobic protein (SH). The G protein interacts with cellular receptors on the host cell membrane to attach the virus particle to the cell surface. The protein consists of a central conserved domain (CCD), two glycosylated mucin-like regions (MLR) and an N-terminal region containing a transmembrane domain and a cytoplasmic domain. Sequencing of the G-gene indicated that the two mucin-like regions flanking the central domain only have a 67% similarity at the nucleotide level between RSV-A and RSV-B and only 53% similarity at the nucleotide level between RSV-A and RSV-B and only 53% similarity at the deduced amino acid levels. Consequently, the two mucin-like regions serve as excellent targets for RSV evolution studies. Both subtypes are further divided into genotypes based on those genetic variations. For RSV-A, the genotypes GA1-7, SAA1-2, NA1-4 and ON1 have been defined, while for RSV-B, the GB1-5, SAB1-4, URU1-2, BA1-12 and THB genotypes are reported. The F protein is responsible for the fusion of the viral envelope with the host membrane. An important side-effect is the fusion of the cell membrane of an infected cell with adjacent cells, resulting in a giant cell with multiple nuclei, better known as a syncytium. The formation of syncytia is recognized as a means to efficiently spread the infection along epithelial surfaces, while minimizing contact with the immune system.

One of the hallmarks of the pathology caused by RSV is increased mucus production in the lungs of infected individuals. Mucus is a gel-like substance that consists of different mucins (MUC), which are high molecular mass, highly glycosylated glycoproteins. Airway mucus protects the epithelial surface from injury through mucociliary clearance, facilitating the removal of foreign particles and chemicals that enter the lung. Twenty-one MUC proteins have been described in humans and are divided in two families: secreted mucins and cell-tethered mucins. The major mucins produced in the airways are MUCSAC and MUCSB as secreted mucins and MUC1, MUC4, MUC16 and MUC20 as membrane-bound mucins.

Genetic variations in the host inflammatory immune responses to RSV have been implicated in the wide range of RSV-related diseases, however, the role of factors intrinsic to the virus itself in contribution to disease severity have only partially been researched.

The wide spectrum of pulmonary manifestations associated with RSV-infections arises from the complex pathogenesis, in which direct virus-induced cytotoxicity, virus-induced immunopathology, genetic constitution and environmental factors play a crucial role but they also complicate an adequate prognosis for a subject infected with RSV. After all, whereas diagnosis of an RSV infection has become quite easy nowadays by the development of rapid point-of-care molecular testing methods, it remains to be elucidated why some infected individuals develop severe disease while others do not. In particular, until today, an estimate about the severity and/or prognosis of an RSV infection at the time of diagnosis of the RSV infection in a subject remains challenging.

### SUMMARY

The present application is directed to an in vitro method for determining the severity and/or prognosis of an RSV infection based on the expression level of one or more mucin genes in respiratory epithelial cells that are in contact with RSV. In particular, the inventors found that an increased mucin expression in respiratory epithelial cells is correlated with a more aggressive RSV, and thereby leads to a more severe RSV infection.

In a first aspect, the present invention is thus directed to an in vitro method for determining the severity and/or prognosis of an RSV infection in a subject based on the expression level of one or more mucin genes in respiratory epithelial cells that are cultured in vitro and exposed to an RSV isolate of the subject. In said aspect, changes in the expression level of said one or more mucin genes are indicative for the severity and/or prognosis of the respiratory syncytial virus infection. In particular, an increased expression level of said one or more mucin genes is indicative for an increased severity and/or worse prognosis of the RSV infection. In a further embodiment, said one or more mucin genes are selected from MUC1, MUC2, MUC4, MUC5AC, MUC5B, MUC6 and MUC13. Even more preferred, the mucin gene is MUC13.

Thus, in one aspect, the present invention provides an in vitro method for determining the severity and/or prognosis of an RSV infection in a subject based on the expression level of MUC1 in respiratory epithelial cells. In another aspect, the present invention provides an in vitro method for determining the severity and/or prognosis of an RSV infection in a subject based on the expression level of MUC2 in respiratory epithelial cells. In also another aspect, the present invention provides an in vitro method for determining the severity and/or prognosis of an RSV infection in a subject based on the expression level of MUC4 in respiratory epithelial cells. In still another aspect, the present invention provides an in vitro method for determining the severity and/or prognosis of an RSV infection in a subject based on the expression level of MUC5AC in respiratory epithelial cells. In a further aspect, the present invention provides an in vitro method for determining the severity and/or prognosis of an RSV infection in a subject based on the expression level of MUC5B in respiratory epithelial cells. In another aspect, the present invention provides an in vitro method for determining the severity and/or prognosis of an RSV infection in a subject based on the expression level of MUC6 in respiratory epithelial cells. In another aspect, the present invention provides an in vitro method for determining the severity and/or prognosis of an RSV infection in a subject based on the expression level of MUC13 in respiratory epithelial cells. Still, in a further aspect, an in vitro method for determining the severity and/or prognosis of an RSV infection in a subject based on the expression level of MUC1, MUC2, MUC4, MUC5AC, MUC5B, MUC6 and/or MUC13, or any combination thereof. In a most preferred aspect, an in vitro method is provided for determining the severity and/or prognosis of an RSV infection in a subject based on the expression level of MUC13 in respiratory cells that are exposed to an RSV isolate of the subject.

The present invention is thus directed to an in vitro method wherein the expression level of one or more mucin genes in respiratory epithelial cells is evaluated to determine the severity and/or prognosis of an RSV infection in a subject.

In one embodiment of the invention, the respiratory epithelial cells are presented in or isolated from a biological sample obtained from the subject. In a further aspect, the biological sample is selected from mucus, sputum, nasopharyngeal aspirate, bronchalveolar aspirate or a bronchoalveolar tissue biopsy.

In another and preferred embodiment of the invention, the respiratory epithelial cells are respiratory epithelial cells that are cultured in vitro and that are exposed to an RSV isolate of the subject. In a further embodiment, said RSV isolate is isolated from a biological sample of the subject; preferably from mucus, sputum, nasopharyngeal aspirate, bronchoalveolar aspirate or a tissue biopsy of the subject. In a further embodiment, said respiratory epithelial cells are derived from an epithelial cell line. In still a further embodiment, the respiratory epithelial cells are cells selected from the HEp-2 cell line, the A549 cell line, the Vero cell line, or the BEAS-2B cell line.

The respiratory epithelial cells in the method of the present invention can further be selected from nasal epithelial cells, pharyngeal epithelial cells, bronchial epithelial cells, and/or lung epithelial cells.

The method of the present invention is based on evaluation of the expression of one or more mucin genes in respiratory epithelial cells. In a further embodiment, the expression level of said one or more mucin genes is determined based on RNA, cDNA, mRNA, and/or protein expression.

The present invention thus allows for determining the severity and/or prognosis of an RSV infection in subject. In a preferred embodiment the subject is a human subject. In still a further preferred embodiment, the subject is a child or an infant with an age below 12 years. In another embodiment, the subject is an adult. In still a further embodiment, the subject is an older adult.

In another aspect, the subject of the present invention can already be diagnosed with an RSV infection. In another aspect, the subject of the present invention is not yet diagnosed with an RSV infection. In still another aspect, the subject of the present invention is not yet diagnosed with an RSV infection but shows the clinical symptoms of an RSV infection.

The RSV infection can be an acute RSV infection. In another embodiment, the RSV infection is a chronic RSV infection.

In another aspect, the RSV infection is selected from pharyngitis, croup, bronchiolitis, pneumonia, or a combination thereof. In still another aspect, the RSV infection is selected from acute pharyngitis, acute croup, acute bronchiolitis, acute pneumonia, or a combination thereof. In still another embodiment, the RSV infection is selected from chronic pharyngitis, chronic croup, chronic bronchiolitis, chronic pneumonia, or a combination thereof.

In another aspect, the present application also provides a method for the treatment of a subject with an RSV infection. Said method comprises determining the severity and/or prognosis of an RSV infection in a subject using the in vitro method as disclosed in any of the previously described embodiments, followed by selection of a therapy based on the observed expression of one or more mucin genes measured in said in vitro method, followed by treatment of the subject with said therapy.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIGS. 1A and 1B: Phylogenetic trees for RSV-A and RSV-B clinical isolates. The phylogenetic trees were constructed with maximum-likelihood with 1000 bootstrap replicates using MEGA X software. The trees are based on a 342 nt and 330 nt fragment of the G protein of RSV-A (FIG. 1A) and RSV-B (FIG. 1B) strains respectively, consisting of the second hypervariable region. Nucleotide sequences of the clinical isolates (indicated with ⋅) were compared to reference strains found on GenBank (indicated with genotype and accession number). The outgroups are represented by prototype strains M11486 for RSV-A and M17213 for RSV-B. Bootstrap values greater than 70% are indicated at the branch nodes and the scale bare represents the number of substitutions per site.

FIGS. 2A-2D: Growth kinetics and infectious virus production in HEp-2 cells. (A-B) HEp-2 cells were infected with clinical isolates and RSV reference strains A2 and B1. Cultures were fixed after 24 h, 48 h and 72 h, permeabilized and stained with polyclonal antibody (pAb) goat-anti-RSV antibody and AF488 donkey-anti-goat (IgG). Nuclei were visualized with DAPI and cultures were analyzed with fluorescence microscopy. RSV positive cells were counted and calculated to the total number of nuclei to reach a percentage of RSV infected cells. (FIG. 2A) Growth kinetics of RSV-A clinical isolates and (FIG. 2B) Growth kinetics of RSV-B clinical isolates. (FIGS. 2C-2D) HEp-2 cells were infected with clinical isolates and RSV reference strains A2 and B1. After 24 h, 48 h and 72 h, supernatants were collected and used for quantification by conventional plaque assay. Data represents mean values±SEM (N=3), significant differences compared to the reference strains are indicated by *p<0.05; ***p<0.001 (two-way ANOVA).

FIGS. 3A-3D: Growth kinetics and infectious virus production in A549 cells. (FIGS. 3A-3B) A549 cells were infected with clinical isolates and RSV reference strains A2 and B1. Cultures were fixed after 24 h, 48 h and 72 h, permeabilized and stained with pAb goat-anti-RSV antibody and AF488 donkey-anti-goat (IgG). Nuclei were visualized with DAPI and cultures were analyzed with fluorescence microscopy. RSV positive cells were counted and calculated to the total number of nuclei to reach a percentage of RSV infected cells. (FIG. 3A) Growth kinetics of RSV-A clinical isolates and (FIG. 3B) Growth kinetics of RSV-B clinical isolates. (FIGS. 3C-3D) A549 cells were infected with clinical isolates and RSV reference strains A2 and B1 . After 24 h, 48 h and 72 h, supernatants were collected and used for quantification by conventional plaque assay. Data represents mean values±SEM (N=3), significant differences compared to the reference strains are indicated by *p<0.05; ***p<0.001 (two-way ANOVA).

FIGS. 4A-4D: Growth kinetics and infectious virus production in BEAS-2B cells. (FIGS. 4A-4B) BEAS-2B cells were infected with clinical isolates and RSV reference strains A2 and B1. Cultures were fixed after 24 h, 48 h and 72 h, permeabilized and stained with pAb goat-anti-RSV antibody and AF488 donkey-anti-goat (IgG). Nuclei were visualized with DAPI and cultures were analyzed with fluorescence microscopy. RSV positive cells were counted and calculated to the total number of nuclei to reach a percentage of RSV infected cells. (FIG. 4A) Growth kinetics of RSV-A clinical isolates and (FIG. 4B) Growth kinetics of RSV-B clinical isolates. (FIGS. 4C-4D) BEAS-2B cells were infected with clinical isolates and RSV reference strains A2 and B1. After 24 h, 48 h and 72 h, supernatants were collected and used for quantification by conventional plaque assay. Data represents mean values±SEM (N=3), significant differences compared to the reference strains are indicated by *p<0.05;**p<0.01 ***;p<0.001 (two-way ANOVA).

FIGS. 5A-5F: Thermal stability profiles at 37° C., 32° C. and 4° C. Clinical isolates, RSV A2 and RSV B1 were aliquoted and exposed to 37° C. (FIGS. 5A-5B), 32° C. (FIGS. 5C-5D) or 4° C. (FIGS. 5E-5F). One aliquot of each was snap frozen at Oh, 24 h, 48 h and 72 h. Aliquots were used for quantification by conventional plaque assay and calculated to the amount at 0 h. Data represents mean values±SEM (N=3), significant differences compared to the reference strains are indicated by *p<0.05;**p<0.01; ***p<0.001 (two-way ANOVA).

FIGS. 6A-6D: The capacity for syncytia formation of clinical isolates. HEp-2 cells were infected with clinical isolates and RSV reference strains A2 and B1 for 2 h, inoculum was replaced by DMEM-10 containing 0.6% Avicel® and incubated for 48 h at 37° C. Afterwards, cells were fixed, permeabilized and stained with pAb goat-anti-RSV and AF488 donkey-anti-goat. Nuclei were visualized with DAPI and cultures were analyzed with fluorescence microscopy. (FIGS. 6A-6B) Mean syncytium size was calculated by counting the number of nuclei in syncytia in three pictures taken at 10× magnification. (FIGS. 6C-6D) Mean syncytium frequency was calculated by dividing the number of syncytial cells by the total number of infected cells. Data represents mean values±SEM (N=3), significant differences compared to the reference strains are indicated by *p<0.05; ***p<0.001 (one-way ANOVA).

FIG. 7: Plaque reduction of the clinical isolates with palivizumab. HEp-2 cells were infected for 2 h with clinical isolates and reference strains that were pre-incubated for 1 h with a palivizumab dilution series. Inoculum was replaced with DMEM-10 containing 0.6% Avicel® and incubated for three days at 37° C. Afterwards, the cells were fixed, stained with palivizumab as primary antibody and goat-anti-human conjugated with HRP, plaques were visualized with chloronapthol. Individual values are plotted as 2log EC50, data represents mean values±SD (N=3).

FIGS. 8A-8G: mRNA levels of mucins 1, 4, 5AC and 5B in infected 549 cells. A549 cells were infected with an MOI of 0.1 of clinical isolates and reference strains for 2 h at 37° C. Inoculum was replaced with DMEM-10 and cells were incubated for 48 h at 37° C. Afterwards, cells were lysed, total RNA was extracted and the expression of MUC1 (FIG. 8A), MUC4 (FIG. 8B), MUC5AC (FIG. 8C), MUC5B (FIG. 8D), MUC2 (FIG. 8E), MUC6 (FIG. 8F) and MUC13 (FIG. 8G) was determined by qRT-PCR. Data represents mean values ±SEM (N=3), statistically significant differences compared to the reference strains are indicated with ***p<0.001 (one-way ANOVA).

FIG. 9: Correlation between MUC13 mRNA expression and “Resvinet” score. Pearson's correlation was used to determine the relationship between the variables ‘relative MUC13 mRNA expression’ and ‘Resvinet socre’.

### DETAILED DESCRIPTION

### Definitions

In the context of the present application, “diagnosis” and “diagnosing” generally includes a determination of a subject's susceptibility to a disease or disorder, a determination as to whether a subject is presently affected by a disease or disorder.

The terms “prognosis” and “prognose” refer to the act or art of foretelling the course of a disease. Additionally, the terms refer to the prospect of survival and recovery from a disease as anticipated from the usual course of that disease or indicated by special features of the individual case.

The term “severity” of a disease refers to the extent of an organ system derangement or physiologic decompensation for a patient. It gives a medical classification such as minor, moderate, major and extreme. The severity of a disease is used to provide a basis for evaluating hospital resource use and to establish patient care guidelines.

The terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptom but has not yet been diagnosed as having it; (b) inhibiting the disease symptoms, i.e. arresting its development; or (c) relieving the disease symptom, i.e. causing regression of the disease or symptom.

The term “biological sample” encompasses a variety of fluid samples, including blood and other liquid samples of biological origin, or tissue samples, or mixed fluid-cell or mixed fluid-tissue samples, obtained from an organism that may be used in a diagnostic or monitoring assay. The term specifically encompasses a clinical fluid or tissue sample, and further includes cell supernatants, cell lysates, serum, plasma, urine, amniotic fluid, biological fluids, tissue biopsies, lavages, aspirates, sputum or mucus. The term also encompasses samples that have been manipulated in any way after procurement, such as treatment with reagents, solubilization, or enrichment for certain components.

RSV is an important viral pathogen in children, immunocompromised and cardiopulmonary diseased patients and the elderly. The wide spectrum of pulmonary manifestations associated with RSV-infections arises from the complex pathogenesis, in which direct virus-induced cytotoxicity, virus-induced immunopathology, genetic constitution and environmental factors play a crucial role but they also complicate an adequate prognosis for a subject infected with RSV. After all, whereas diagnosis of an RSV infection has become quite easy nowadays by the development of rapid point-of-care molecular testing methods, it remains to be elucidated why some infected individuals develop severe disease while others do not. In particular, until today, an estimate about the severity and/or prognosis of an RSV infection at the time of diagnosis of the RSV infection in a subject remains challenging.

With the present invention, an in vitro method for determining the severity and/or prognosis of a respiratory syncytial virus infection in a subject has been developed. The method according to the invention makes it possible to make an estimate of the severity and/or prognosis of an RSV infection at the time of diagnosis of said RSV infection. As such, treatment options can be adapted to the complexity and severity of the RSV infection.

The present invention thus relates to an in vitro method for determining the severity and/or prognosis of a respiratory syncytial virus infection based on the expression level of one or more mucin genes. In particular, the expression level of one or more mucin genes is evaluated in respiratory epithelial cells that are cultured in vitro and that are exposed to an RSV isolate of the subject. In a further embodiment, the one or more mucin genes are selected from MUC1, MUC2, MUC4, MUC5AC, MUC5B, MUC6 and MUC13. In the present method, an increased expression level of said one or more mucin genes is indicative for an increased severity and/or worse prognosis of the RSV infection. In an even more preferred embodiment, the in vitro method according to the invention is based on the expression level of MUC13 in respiratory epithelial cells.

The inventors of the present application found that based on the expression level of one or more of mucin genes in respiratory epithelial cells, the severity and/or prognosis of a respiratory syncytial virus (RSV) infection can be determined. In particular, a correlation was found between the expression level of one or more mucin genes, in particular MUC13, in respiratory epithelial cells that were exposed to an RSV or a mixture of RSVs and the severity and/or prognosis of such an RSV infection in a subject.

In one aspect of the invention, the expression level of one or more mucin genes is determined in respiratory epithelial cells present in a biological sample of a subject. Said biological sample can be selected from mucus, sputum, nasopharyngeal aspirate, bronchoalveolar aspirate, or even tissue biopsies from airway epithelial cells. The biological sample can be taken from the subject using a nasal swab, a pharyngeal swab, a throat swab, or a lavage.

Respiratory epithelial cells can be isolated from the biological sample using any suitable technology known to the skilled person.

In another and most preferred aspect of the invention, the expression level of one or more mucin genes is determined in respiratory epithelial cells that are cultured in vitro. Said respiratory epithelial cells are then exposed to an RSV isolate of the subject. In a further embodiment, the RSV isolate is isolated from a biological sample of the subject. Said biological sample can be selected from mucus, sputum, nasopharyngeal aspirate, bronchoalveolar aspirate, or even tissue biopsies from airway epithelial cells. The biological sample can be taken from the subject using a nasal swab, a pharyngeal swab, a throat swab, or a lavage. In the context of this aspect of the invention, an RSV isolate is an RSV or a mixture of RSVs that is present in the biological sample of the subject. Said RSV or several RSVs will induce changes in the expression level of one or more mucin genes in the respiratory epithelial cells that are exposed to the RSV isolates. In a particular embodiment, the respiratory epithelial cells to which the RSV isolates are exposed are derived from an epithelial cell line. In still a further embodiment, the respiratory epithelial cells are cells selected from the HEp-2 cell line, the A549 cell line, the Vero cell line, or the BEAS-2B cell line.

As said, the method according to this invention allows for determining the severity and/or prognosis of an RSV infection based on the expression of one or more mucin genes, in particular MUC1, MUC2, MUC4, MUC5AC, MUC5B, MUC6 and MUC13 in respiratory epithelial cells, even more in particular based on MUC13 expression in respiratory epithelial cells. Said respiratory epithelial cells can be selected from nasal epithelial cells, pharyngeal epithelial cells, bronchial epithelial cells, and/or lung epithelial cells.

The method of the present invention is based on evaluation of the expression of one or more mucin genes in respiratory epithelial cells, preferably respiratory epithelial cells that are cultured in vitro and subsequently exposed to an RSV isolate of the subject. In a further embodiment, the expression level of said one or more mucin genes is determined based on RNA, cDNA, mRNA, and/or protein expression. In a specific embodiment, the in vitro method is based on the mRNA expression of one or more mucin genes. mRNA levels can be determined by polymerase chain reaction, real-time polymerase chain reaction, reverse transcriptase polymerase chain reaction, hybridization, probe hybridization, or quantitative gene expression arrays. In another embodiment, the in vitro method is based on the protein expression of one or more mucin genes. Protein expression levels can be determined by western blotting, or immune-based technologies, such as enzyme-linked immunosorbent assay (ELISA), immune-chromatography, Luminex assays, CyTOF, or immunofluorescence assays. In still another further embodiment, the method of the present invention is based on a combination of mRNA and protein expression levels of one or more mucin genes.

Also in the context of this invention, a “change in expression” is meant an upregulation of one or more selected genes in comparison to the reference or control; a downregulation of one or more selected genes in comparison to the reference or control; or a combination of certain upregulated genes and certain down regulated genes. In the context of the present application, the reference or control is used to indicate a healthy or unaffected subject or sample isolated from a healthy or unaffected subject.

The present invention thus allows for determining the severity and/or prognosis of an RSV infection in subject. In a preferred embodiment the subject is a human subject. In still a further preferred embodiment, the subject is a child or an infant with an age under 12 years. In another embodiment, the subject is an adult. In still a further embodiment, the subject is an older adult.

In another aspect, the subject of the present invention can already be diagnosed with an RSV infection. In another aspect, the subject of the present invention is not yet diagnosed with an RSV infection. In still another aspect, the subject of the present invention is not yet diagnosed with an RSV infection but shows the clinical symptoms of an RSV infection.

The RSV infection can be an acute RSV infection. In another embodiment, the RSV infection is a chronic RSV infection.

In another aspect, the RSV infection is selected from pharyngitis, croup, bronchiolitis, pneumonia, or a combination thereof. In still another aspect, the RSV infection is selected from acute pharyngitis, acute croup, acute bronchiolitis, acute pneumonia, or a combination thereof. In still another embodiment, the RSV infection is selected from chronic pharyngitis, chronic croup, chronic bronchiolitis, chronic pneumonia, or a combination thereof.

In the present invention, an in vitro method is provided for determining the severity and/or prognosis of an RSV infection in a subject based on the expression level of one or more mucin genes in respiratory epithelial cells. In a particular embodiment, the inventors show that the expression of said one or more mucin genes in respiratory epithelial cells correlate with an increased presence of clinical symptoms of RSV infection, more specifically, the inventors found that an increased expression level of said one or more mucin genes correlates with the Resvinet scale [1]. The Resvinet scale is a properly validated system allowing objective categorization of infants with acute respiratory infections, such as RSV infections. The Resvinet scale is based on seven parameters (feeding intolerance, medical intervention, respiratory difficulty, respiratory frequency, apnoea, general condition and fever) that are assigned different values (from 0 to 3) for a total of 20 points. An increased score on the Resvinet scale indicates that the subject suffers from a more severe respiratory infection.

In another aspect, the present application also provides a method for the treatment of a subject with an RSV infection. Said method comprises determining the severity and/or prognosis of an RSV infection in a subject using the in vitro method as disclosed in any of the previously described embodiments, followed by selection of a therapy based on the observed expression of one or more mucin genes measured in said in vitro method, followed by treatment of the subject with said therapy. In a preferred embodiment, the one or more mucin genes are selected from MUC1, MUC2, MUC4, MUC5AC, MUC5B, MUC6 and MUC13. Preferably, the expression of MUC13 in respiratory cells is evaluated. Based on the observed expression levels of the one or more mucin genes in the respiratory cells exposed to an RSV isolate of the subject, an appropriate therapy is selected and the subject is treated with said therapy. In another embodiment, treatment of individuals that are at an early stage of infection is envisaged. Said individuals are known to develop severe RSV disease and are diagnosed with any of the methods described herein. Treatment options can for example be treatment with antiviral agents or RSV-specific antibodies or antibody-like molecules. Other possible types of therapy include the use of agents that modulate mucin expression and/or mucin production. Said agents can be selected from, but are not limited to, mucus regulators, mucolytics, MARCKS blockade, heat shock protein-70 inhibitors, soluble NSF attachment protein receptors cleavage, Munc inhibitors, P2Y2 agonists/antagonists, or macrolide antibiotics.

### EXAMPLES

### Materials and Methods

### Cells and Viruses

The HEp-2, A549 and Vero cell lines were obtained from and cultured to the instructions of ATCC. BEAS-2B cell line was a generous gift from dr. Ultan F. Power (Queens University Belfast, Ireland). All cells were cultured in Dulbecco's modified Eagle medium containing 10% inactivated fetal bovine serum)(DMEM¹⁰) (Thermo Fisher Scientific). RSV reference strains A2 and B1 were obtained from BEI resources, RSV A2 was cultivated in HEp-2 cells as described by Van der Gucht W. et al [2] and RSV B1 was cultivated on Vero cells in medium containing 2% inactivated fetal bovine serum (iFBS) until cytopathic effect (CPE) was visible throughout the flask. Virus was collected as described for A2 and quantified in a conventional plaque assay on HEp-2 as described by Schepens B. et al [3]. Briefly, HEp-2 cells were seeded at a concentration of 175,000 cells/ml in clear 96 well plates (Falcon) 1 day prior to infection. Cells were washed with DMEM without iFBS)(DMEM⁰) and infected with 50 μl of a 1/10 dilution series made in DMEM⁰. Cells were incubated for 2 h at 37° C. after which the inoculum was replaced by DMEM¹⁰ containing 0.6% avicel (FMC biopolymer) and incubated for 3 additional days at 37° C., 5% CO₂. Afterwards, cells were washed with PBS, fixed with 4% paraformaldehyde solution and stained with palivizumab (leftovers provided by the department of Pediatrics, Antwerp University Hospital) and goat-anti human secondary IgG conjugated with horseradish peroxidase (HRP) (Thermo Fisher Scientific) and visualized using chloronaphtol solution (Thermo Fisher Scientific).

### Virus Isolation from Clinical Samples

This study was approved by the ethical committee of the Antwerp University Hospital and the University of Antwerp (Ser. No. 16/46/491). Mucus was collected from children showing symptoms of an RSV-related bronchiolitis during the winter seasons of 2016-2017 and 2017-2018 after parental consent was given. The mucus was extracted by a nasal swab and/or a nasopharyngeal aspirate, which were stored at 4° C. for less than 10 h. One day prior to mucosal extraction, HEp-2 cells were seeded at a concentration of 175 000 cells/ml in a clear 96 well plate (Falcon). Samples were vortexed for 1 minute with glass beads (Sigma-Aldrich) before inoculating HEp-2 cells with 50 μl of a ¼ dilution series of the sample, made in DMEM without iFBS) (DMEM⁰). After 2 h of incubation with the inoculum, 50 μl of DMEM containing iFBS, antibiotics (penicillin/streptomycin (life technologies), moxifloxacin (Sigma-Aldrich)) and anti-fungals (Fungizone)(Sigma-aldrich) was added to obtain a final concentration of DMEM with 2% FBS. Plates were incubated for 7 days at 37° C. and 5% CO₂. After 7 days, the plates were checked for syncytia formation and 50 μl of the well with the lowest concentration of original sample but still presenting CPE, was transferred to a newly seeded plate, following the same protocol. After another 7 days, the wells were rechecked for syncytia formation. A total of 250 μl from wells with syncytia was transferred to a freshly seeded T25, which was left until cytopathic effects were visible throughout the flask. Supernatant was collected, centrifuged for 10 min at 1000× g, aliquoted, snap frozen in liquid nitrogen and labelled passage 0. Virus obtained from these clinical samples was propagated until passage 3 on HEp-2 cells to obtain a plaque forming unit (PFU) high enough to perform the following experiments. One sample did not propagate efficiently on HEp-2 cells and was propagated for 3 passages on Vero cells until a high enough PFU was reached.

### RSV-A and RSV-B Subtyping

RNA for subtyping was extracted from passage 0 virus using the QIamp viral RNA extraction mini kit (QlAgen) following the manufacturer's instructions. A multiplex reaction mix was made with superscript III platinum one-step quantitative kit (Thermo Fisher Scientific) in a final volume of 25 μl containing 5 μl RNA, 12.5 μl PCR master mix, 1 μl superscript RT/Platinum Taq polymerase and 2.5 μl of a pre-mixed primer/probe solution. This solution contains a final concentration of 5 μM of each primer and 1 μM of each probe. The primers for RSV-A are located in the L gene (RSVQA1: 5′-GCT CTT AGC AAA GTC AAG TTG AAT GA-3′ (SEQ ID NO:1) and RSVQA2: 5′-TGC TCC GTT GGA TGG TGT AAT-3′ (SEQ ID NO:2), RSVQA probe: 5′-HEX/ACA CTC AAC AAA GAT CAA CTT CTG TCA TCC AGC-′3-IABkFQ (SEQ ID NO:3) wherein ZEN is inserted after ACA CTC AAC in the probe) and the primers for RSV-B are located in the N gene (RSVQB1: 5′-GAT GGC TCT TAG CAA AGT CAA GTT AA-3′ (SEQ ID NO:4) and RSVQB2: 5′-TGT CAA TAT TAT CTC CTG TAC TAC GTT GAA-3′ (SEQ ID NO:5), RSVQB probe: 5′-RTEX615/TGA TAC ATT AAA TAA GGA TCA GCT GCT GTC ATC CA-′3-BHQ_2 (SEQ ID NO:6)). Reaction was run on a Real-time PCR machine (Stratagene, Mx3000P, Thermo Fisher Scientific) with the following program: 50° C. for 30 min, 94° C. for 5 min followed by 45 cycles of 15 s at 94° C. and 1 min at 55° C. Ct values below 40 were counted as positive.

### Nucleotide Sequencing and Phylogenetic Analysis

Viral RNA was extracted using the QIAmp viral RNA mini kit (Qiagen) according to the instructions provided by the manufacturer. Viral RNA of the G-gene was transcribed to cDNA and amplified using the One-step RT-PCR kit (Qiagen) and the following primers as described by L. Houspie et al. [4]. For RSV-A, the forward primer G267FW (5′ ATG CAA CAA GCC AGA TCA AG 3′ (SEQ ID NO:7) and reverse primer F164RV (5′ GTT ATC ACA CTG GTA TAC CAA CC 3′ (SEQ ID NO:8)) were used, for RSV-B, the forward primer BGF (5′ GCA GCC ATA ATA TTC ATC ATC TCT 3′ (SEQ ID NO:9)) and reverse primer BGR (5′ TGC CCC AGR TTT AAT TTC GTT C 3′ (SEQ ID NO:10)) were used. Primers were added to the reaction mix consisting of 10 μl 5× RT-PCR buffer, 2 μl dNTP, 2 ml enzyme, 20 μH₂O to a final concentration of 30 pmol. 10 μl RNA extract was added to the reaction mix. The PCR was performed in a thermocycler (Unocycler, VWR) following the given program: 30 min at 50° C. for the Reverse Transcription step, 15 min at 95° C. for PCR activation, 40 amplification cycles consisting of 30 s at 95° C., 1 min at 55° C. and 1 min at 72° C. followed by a final extension step for 10 min at 72° C. The amplified cDNA was subjected to a 1% agarose gel electrophoresis, visualized with Gelgreen™ (VWR) to determine the length. Amplified cDNA was delivered to the VIB Genetic service facility (University of Antwerp) for PCR cleanup and DNA sequencing with the following primers as described by L. Houspie et al [4]: in addition to the PCR amplification primers, for RSV-A: G516R (5′ GCT GCA GGG TAC AAA GTT GAA C 3′ (SEQ ID NO:11)) and G284F (5′ ACC TGA CCC AGA ATC CCC AG 3′ (SEQ ID NO:12)) and for RSV-B: BGF3 (5′ AGA GAC CCA AAA ACA CYA GCC AA 3′ (SEQ ID NO:13)) and BGR3 (5′ ACA GGG AAC GAA GTT GAA CAC TTC A 3′ (SEQ ID NO:14)) were provided for sequencing. Sequences were annotated in Snapgene and contigs were built in Bioedit with the CAP3 application. Multiple sequence alignments from reference strains and contigs and phylogenetic trees were constructed in MEGA X using the maximum likelihood method.

### Viral Replication Kinetics

HEp-2, A549 and BEAS-2B cells were seeded at a concentration of 175 000 cells/ml in black CELLSTAR® 96 well plates with a μclear® flat bottom suitable for fluorescence microscopy (Greiner-bio one) 1 day prior to inoculation. Briefly before inoculation, the cells were washed with DMEM⁰, followed by inoculation. Clinical RSV and RSV-A2 were diluted to infect the cells at a multiplicity of infection (MOI) of 0.01. Virus was left to adhere for 2 h at 37° C., 5% CO₂ and replaced with DMEM¹⁰. Cells were fixed with 4% paraformaldehyde after 24 h, 48 h and 72 h, permeabilized and stained with palivizumab followed by goat anti-human secondary antibody conjugated with Alexa Fluor 488 (AF488) (Thermo Fisher Scientific) and additional DAPI nucleus staining (Sigma-Aldrich).

### Infectious Virus Production

HEp-2, A549 and BEAS-2B cells were seeded at a concentration of 200 000 cells/ml in 24 well plates 24 h prior to infection. Briefly, before infection, cells were washed with DMEM⁰ and afterwards infected with clinical isolates and RSV A2 and RSV B1 at an MOI of 0.01. Supernatant was collected after 24 h, 48 h and 72 h, aliquoted, snap frozen and stored at −80° C. Supernatant was quantified using a conventional plaque assay on HEp-2 cells as described above.

### Thermal Stability Assay

Aliquots of clinical isolates and RSV A2 and RSV B1 were thawed and diluted in DMEM⁰ to obtain a starting concentration of 1×10⁵ PFU/ml and re-aliquoted. Immediately after aliquotation, one aliquot of each sample was snap frozen in liquid nitrogen as T0. The other aliquots were stored at 4° C., at 32° C. or at 37° C. for 24 h, 48 h and 72 h, snap frozen in liquid nitrogen and stored at −80° C. until quantification was performed. A conventional plaque assay on HEp-2 cells as described earlier was used to quantify the remaining PFU in each aliquot.

### Cell-to-Cell Fusion Assay

24 h prior to inoculation, HEp-2 cells were seeded at a concentration of 175 000 cells/ml in black CELLSTAR® 96 well plates with a μclear® flat bottom suitable for fluorescence microscopy (Greiner bio-one). Cells were inoculated with clinical RSV and RSV-A2 at a MOI of 0.05 for 2 h at 37° C., 5% CO₂. After 2 hours, the inoculum was removed and replaced by DMEM¹⁰ containing 0.6% Avicel (FMC biopolymer). After 48 h cells were washed with PBS, fixed with 4% paraformaldehyde solution, permeabilized and stained with palivizumab followed by goat anti-human secondary antibody conjugated with AF488 (Thermo Fisher Scientific). DAPI staining was performed to stain the nuclei (Sigma-Aldrich).

### Plaque Reduction Assay

The plaque reduction assay was performed as described by Leemans A. et al [5]. Briefly, HEp-2 cells were seeded at a concentration of 175 000 cells/ml in a clear 96 well plate (Falcon) 24 h prior to inoculation. Palivizumab was diluted 1:40 and further in a 1:2 dilution series, which was incubated with diluted virus for 1 h at 37° C., 5% CO₂. Afterwards, the cells were washed briefly with DMEM⁰, and inoculated with 50 μl of the virus-antibody solution for 2 h at 37° C., 5% CO₂. Then, the inoculum was replaced with DMEM¹⁰ containing 0,6% avicel (FMC biopolymer). The plates were incubated for 3 days at 37° C., 5% CO₂, washed with PBS and fixed with 4% paraformaldehyde solution. The cells were permeabilized, stained with palivizumab antibody followed by goat anti-human IgG conjugated with horseradish peroxidase (HRP) and colored using chloronaphtol solution (Thermo Fisher Scientific).

### Mucin mRNA Expression Assay

A549 cells were seeded at a concentration of 200 000 cells/ml in 24 well plates 24 h prior to inoculation (Greiner bio-one). Cells were infected with a MOI of 0.1 for 2 h at 37° C., 5% CO₂. After 2 h, inoculum was replaced by DMEM¹⁰ and was incubated for an additional 48 h. After 48 h, cell supernatant was collected, spun down at 1000 xg for 15 minutes and only the pellet was kept. The still adherent cells were lysed with lysis buffer from the nucleospin kit (MN) and added to the pellet. The solution was pipettet up and down several times and frozen at −80° C. until extraction was performed. RNA isolation was done following manufacturer's instructions of the nucleospin RNA kit (MN). Concentrations were evaluated using the Nanodrop® (Thermo Fisher Scientific) and 1 μg of RNA was used to convert to cDNA using the SensiFast™ cDNA synthesis kit (Bioline). Relative gene expression was determined with the GoTaq qPCR master mix (Promega) with SYBR Green Fluorescence detection on a QuantStudie 3 Real-time PCR instrument (Thermo Fisher Scientific). Standard QuantiTect primers available from Qiagen were used for GAPDH (QT00079247), β-actin (QT00095431), MUC1 (QT00015379), MUC4 (QT00045479), MUC5AC (QT00088991) and MUC5B (QT01322818). Analysis and quality control were performed using qbase+software (Biogazelle), relative expression of the target genes was normalized to the expression of the housekeeping genes GAPDH and β-actin.

### F-Gene Nucleotide Sequencing

Viral RNA of the F-gene was transcribed to cDNA and amplified using the One-step RT-PCR kit (Qiagen) and the following primers as described by L. Tapia et al. [6]. For the F-gene, four primers were used in pairs to transcribe and amplify the F-gene in two segments, F1 and F2. For F1, the forward primer RSVAB_F1FW (5′ GGC AAA TAA CAA TGG AGT TG 3′ (SEQ ID NO:15)) and reverse primer RSVAB_F1RV (5′ AAG AAA GAT ACT GAT CCT G 3′ (SEQ ID NO:16)) were used. For F2 the forward primer RSVAB F2FW (5′ TCA ATG ATA TGC CTA TAA CA 3′ (SEQ ID NO:17)) and RSVAB F2RV (5′ GGA CAT TAC AAA TAA TTA TGA C 3′ (SEQ ID NO:18)) were used. Both primer sets are the same for RSV-A and RSV-B strains. Primers were added to the reaction mix consisting of 10 μ× RT-PCR buffer, 2 μl dNTP, 2 ml enzyme, 20 μl H₂O to a final concentration of 30 pmol. 10 μl RNA extract was added to the reaction mix. The PCR was performed in a thermocycler (Unocycler, VWR) with the program: 30 min at 50° C. for the RT step, 15 min at 95° C. for PCR activation, five amplification cycles consisting of 30 s at 95° C., 30 s at 48° C. and 1 min at 72° C. followed by 35 amplification cycles consisting of 30 s at 95° C., 30 s at 55° C. and 1 min at 72° C., and a final extension step for 10 min at 72° C. The length of the amplified cDNA was verified with 1% agarose gel electrophoresis and visualized with Gelgreen™ (VWR). Amplified cDNA was delivered to the VIB Neuromics support facility (University of Antwerp) for PCR cleanup and DNA sequencing with the same primers. Sequences were annotated in SnapGene and contigs were built in BioEdit with the CAP3 application. Multiple sequence alignments from contigs were constructed in MEGA X using Muscle.

### Fluorescence Microscopy and Image Analysis

Fluorescence photographs were acquired using an Axio Observer inverted microscope and a Compact Light source HXP 120C with filter set 49, 10 and 20 for blue, green and red fluorophores respectively (Zeiss). Image analysis was done using Zeiss ZEN 2.3 blue edition imaging software and ImageJ version 2.0.0-rc-43/1.50e. Calculations were made in Excel for Mac and Graphpad Prism 6.

### Statistical Analysis

Data for viral growth kinetics, infectious virus production and thermal stability are presented as means (±SEM) of the indicated independent repeats. To determine the significance between the clinical isolates and the reference (A2 or B1), data was analyzed with a two-way ANOVA. Fusion data and MUC expression represents means (±SEM), significance was calculated between the clinical isolates and their references with a one-way ANOVA. Data for plaque reduction represents means (±SD), significance was calculated between clinical isolates and references with a one-way ANOVA. Calculations were done using Graphpad Prism 6.

### Results

Clinical Samples and Detection of RSV

Nasal swabs and nasopharyngeal aspirates were obtained from one patient in December of 2016 and from 24 patients between October and January 2017-2018. RSV-A was detected in one sample of 2016-2017 and in 11 samples of 2017-2018. RSV-B was also detected in 11 samples of the 2017-2018 season. Of the remaining two RSV-negative samples, one tested positive for human metapneumovirus (hMPV), one remained negative for RSV, hMPV and Rhinovirus 1. HEp-2 cells were infected with the samples on the day of the aspiration of secretions or the day afterwards, without freezing the samples. After two weeks of incubation, 11 samples did not result in syncytia formation or positive fluorescent staining in either the nasal swab culture or the aspirate culture and were therefore not used in any of the following assays. Cultures that showed syncytia formation were used to grow the virus on HEp-2 cells. One sample was further grown on Vero cells since no significant titers could be reached growing the virus on HEp-2 cells. The BE/ANT-A11/17 strain was deposited on Aug. 23, 2019 at the Belgian Co-ordinated Collection of Micro-Organisms (BCCM) with accession number LMBP 11505.

TABLE 1
Overview of clinical isolates and viruses used in experiments,
with subtyping results and cell type used for propagation

| NAME: | SUBTYPE: | GROWN ON: |
|---|---|---|
| BE/ANT-A1/16 | RSV-A | HEp-2 |
| BE/ANT-B2/17 | RSV-B | HEp-2 |
| BE/ANT-A7/17 | RSV-A | HEp-2 |
| BE/ANT-A8/17 | RSV-A | HEp-2 |
| BE/ANT-A10/17 | RSV-A | HEp-2 |
| BE/ANT-A11/17 | RSV-A | HEp-2 |
| BE/ANT-A12/17 | RSV-A | HEp-2 |
| BE/ANT-B13/17 | RSV-B | HEp-2 |
| BE/ANT-B15/17 | RSV-B | HEp-2 |
| BE/ANT-A18/17 | RSV-A | HEp-2 |
| BE/ANT-B20/17 | RSV-B | Vero |
| BE/ANT-A21/17 | RSV-A | HEp-2 |
| RSV A2 | RSV-A | HEp-2 |
| RSV B1 | RSV-B | Vero |

### Phylogenetic Analysis

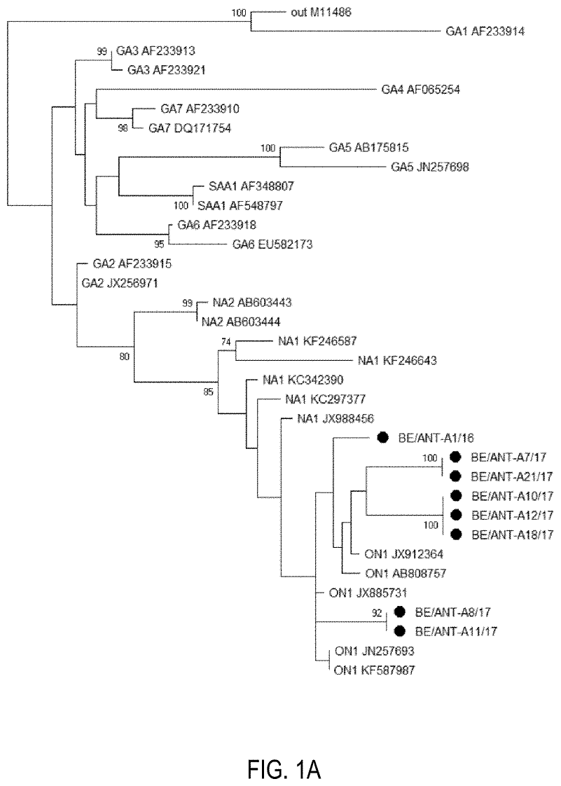

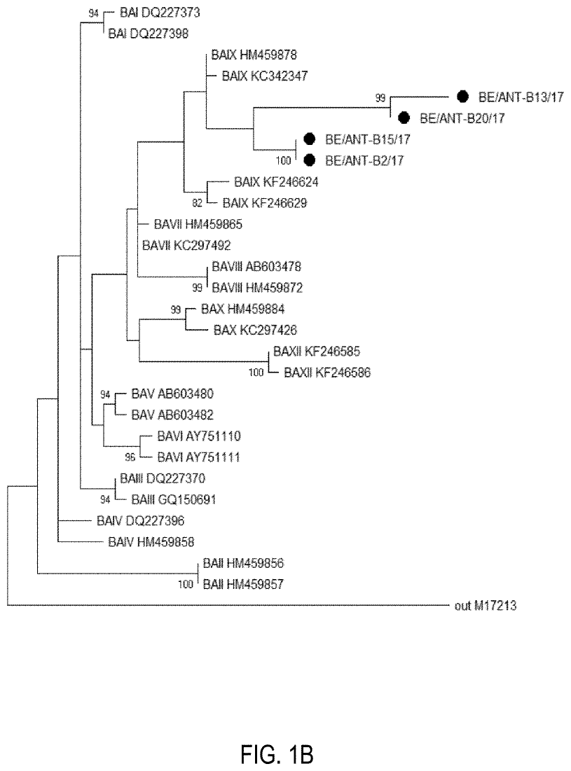

Sequences of the G-gene of all samples were obtained and aligned with previously reported representative sequences from GenBank. The phylogenetic trees of RSV-A and RSV-B sequences were setup (FIG. 1).

All RSV-A sequences cluster within the ON1 genotype that contains a 72 nt duplication and all RSV-B sequences contain a 60 nt duplication in the G-gene, assigning them to the BA genotype, further differentiated into the BAIX genotype.

### G Protein Sequence Analysis

The nucleotide sequence of the G-gene of each clinical isolate was determined and translated to their corresponding in-frame protein sequences by aligning them to the RSV A2 protein sequence in GenBank. Sequences were annotated to the corresponding domains of the G protein sequence: the N-terminal domain (NT), the transmembrane domain (TM), both mucus-like regions (MLR), the central conserved domain (CCD) and the heparin binding domain (HBD). All sequences of recent RSV-A clinical isolates differ from the RSV A2 sequence in 32 amino acids, all spread throughout both MLRs, confirming the use of these regions in phylogeny studies (data not shown). Clinical isolates differ from each other as well in 19 amino acid residues. BE/ANT-A1/16 contains three unique amino acids that are not found in the other clinical isolates, whereas mutations in the clinical isolates obtained in the winter of 2017 are also observed in other clinical isolates. Analysis indicated that sequences of BE/ANT-A7/17 and BE/ANT-A21/17 are very much alike, as well as the G protein sequences of BE/ANT-A10/17, BE/ANT-Al2/17 and BE/ANT-A18/17. Sequences BE/ANT-A8/17 and BE/ANT-A11/17 are also very similar, which is indicated by the phylogenetic analysis. The 72 nt duplication in the MLR-II is present in all clinical isolates starting from amino acid residue 204 to residue 207. Sequences of RSV-B isolates are aligned to the sequence of RSV B1 and all clinical isolates differ from RSV B1 in 21 residues spread out through the MRLs (data not shown). Ten residues are different between the clinical isolates themselves, mainly in the MLRs but also in the HBD. All isolates contain the 60 nt duplication in the MLR-II and a sequence deletion of three residues at the end of MLR-I. Sequences of BE/ANT-B2/17 and BE/ANT-B15/17 are mainly similar, as are BE/ANT-B13/17 and BE/ANT-B20/17.

### Viral Replication Kinetics

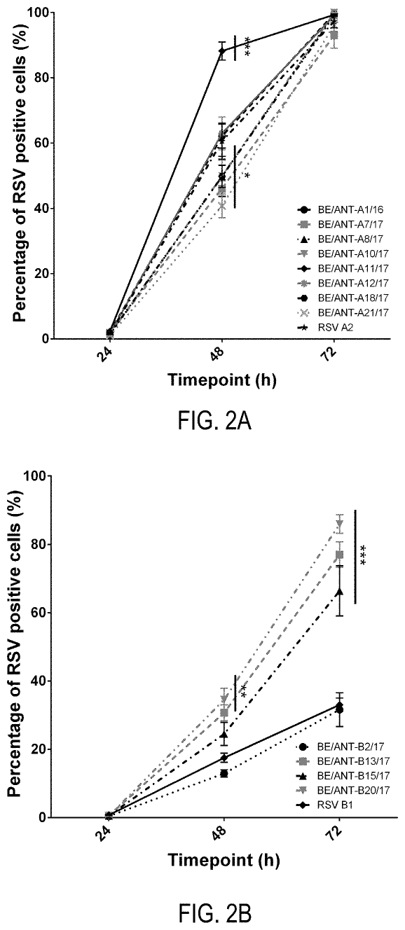

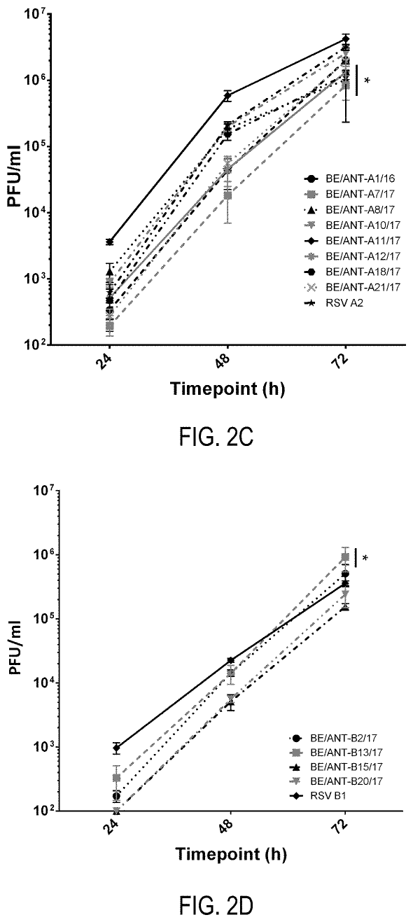

To study the dynamics of viral infection, viral replication kinetics and infectious virus production were assessed in HEp-2, A549 and BEAS-2B cells. Cells were infected for 24 h, 48 h and 72 h with a MOI of 0.01, fixed, fluorescently stained and analyzed with fluorescence microscopy to evaluate viral replication kinetics. Infectious virus production was evaluated through the collection of supernatants after 24 h, 48 h and 72 h post-infection with an MOI of 0.01. Supernatant was snap frozen and used for quantification through plaque assay. Viral replication kinetics in HEp-2 cells for RSV-A (FIG. 2A) strains yielded one strain (BE/ANT-A11/17) that resulted in significantly higher percentages of RSV-infected cells after 48 h compared to RSV A2. The BE/ANT-A11/17 also produced more infectious virus particles after 24 h post inoculation (p.i.) compared to all other strains (FIG. 2C). Three strains (BE/ANT-A21/17, BE/ANT-A7/17, BE/ANT-A8/17) replicated more slowly than the RSV A2 at 48 h but a fully infected culture was observed after 72 h of infection. The RSV-B strains (FIG. 2B and D) showed two strains grown on HEp-2 cells (BE/ANT-B13/17, BE/ANT-B15/17) and one strain grown on Vero cells (BE/ANT-B20/17) that resulted in significantly more infected cells at 72 h than the reference B1 , whereas just one strain (BE/ANT-B2/17) seemed to result in comparable infection as the B1. Infectious virus production of RSV-B shows that even though the BE/ANT-B20/17 and BE/ANT-B15/17 reach a very high percentage of infected cells, significantly less infectious particles are produced compared to the other strains, suggesting that the particles may not be efficiently released in the supernatant and remain more cell-associated.

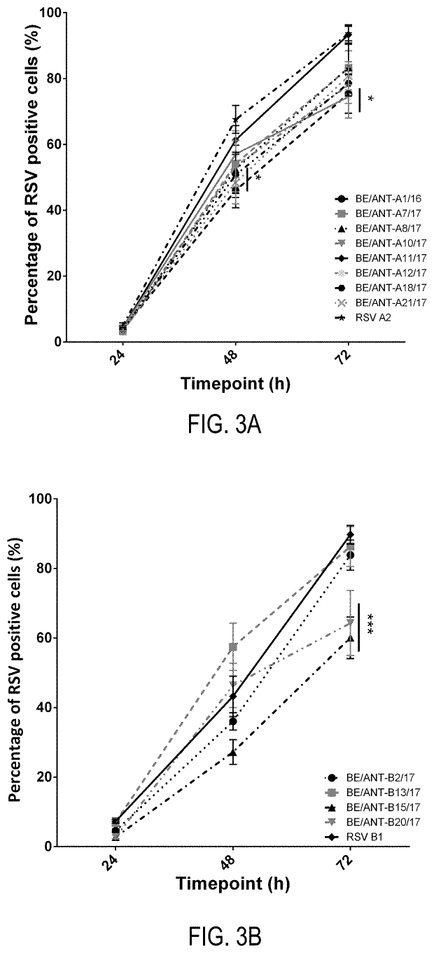

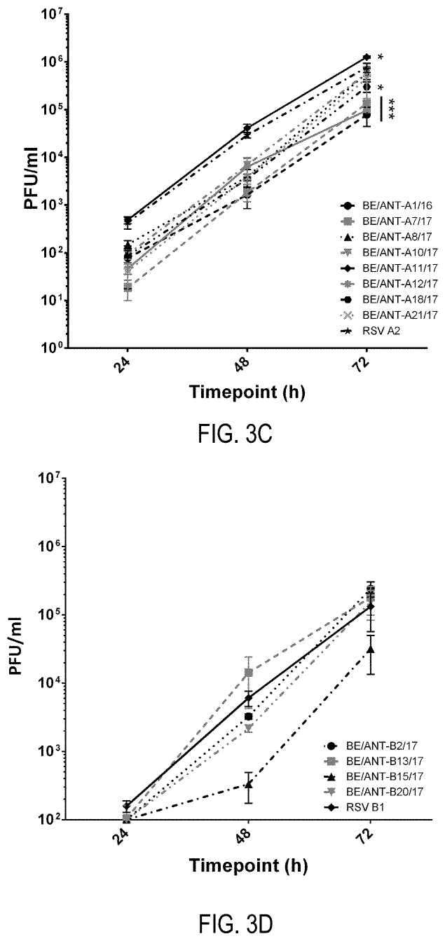

The same experiment was repeated in the A549 (FIG. 3) cell line in which for the RSV-A isolates (FIG. 3A), the RSV A2 shows the highest percentage of infected cells, followed closely by the BE/ANT-A11/17, performing only slightly less than in the HEp-2 cells. The aforementioned strains also were the ones that produced the highest amounts of infectious virus in A549 cells (FIG. 3C). Whereas in HEp-2 cells both the BE/ANT-B13/17 and BE/ANT-B20/17 isolates perform better than the RSV B1, results of A549 replication kinetics suggest that the BE/ANT-B13/17 and BE/ANT-B2/17 strains reach similar infection rates (FIG. 3B). The BE/ANT-B20/17 reached about 50% infection after 48 h but the infection then seemed to flatten out towards 72 h, resulting in a significant difference with infection rates of the RSV B1. Interestingly, the isolate BE/ANT-B2/17, which did not efficiently infected HEp-2 cells now reached a near 100% infection in 72 h. Unsurprisingly, the BE/ANT-B15/17 achieved again the lowest number of infected cells and levels of virus production in A549 cells (FIG. 3D).

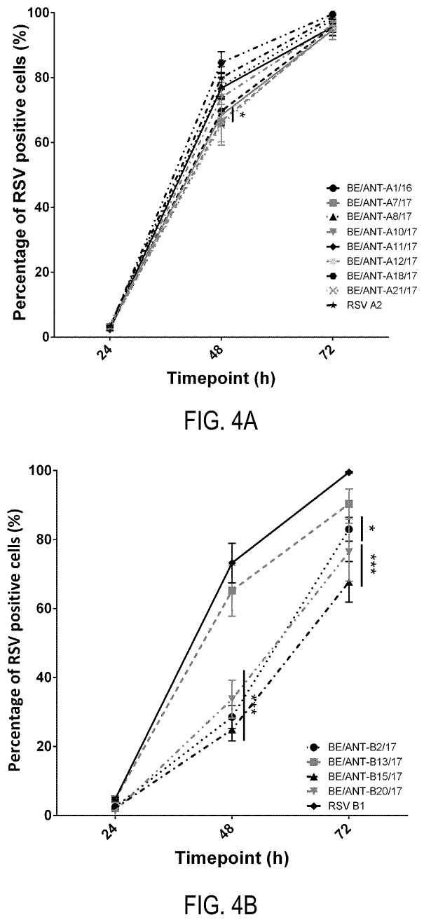

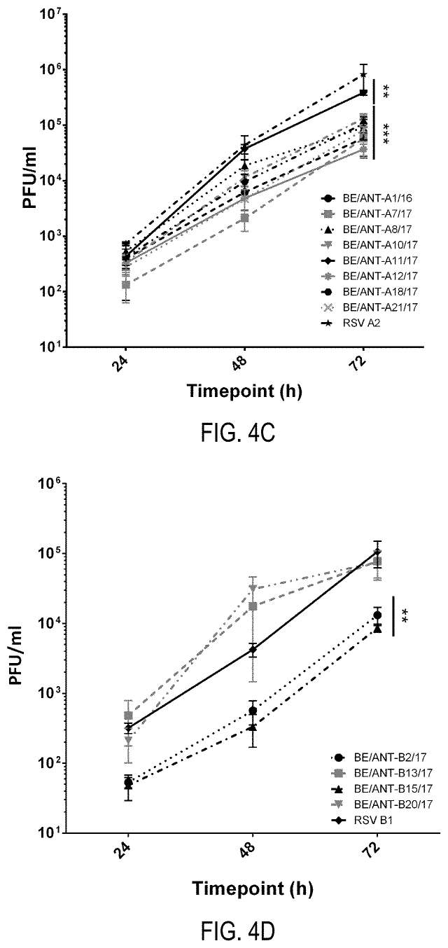

As the BEAS-2B cell line is also a highly permissive cell line for RSV infection and widely used, we also assessed viral growth and production kinetics in this cell line (FIG. 4). For all RSV-A clinical isolates, no major differences were observed after 48 h and 72 h of infection in percentage of infected cells (FIG. 4A). After 72 h of infection, the amount of viable particles released by the cells was the highest for RSV A2 and clinical isolate BE/ANT-A11/17. Larger differences were observed between the clinical isolates of the RSV-B subtype (FIG. 4B). BE/ANT-B13/17 reached percentages and viable particle production that were comparable to RSV-B1 (FIG. 4B and 4D). Isolates BE/ANT-B2/17 and BE/ANT-B15/17 had the lowest infection rates and infectious virus production in both this cell line as well as in the HEp-2 cells (FIG. 4B and 4D).

Overall, clinical isolate BE/ANT-A11/17 replicated very efficiently in all cell lines, and remarkably, achieving even higher infection rates in the HEp-2 cell line than the RSV A2. Also, two clinical isolates of the RSV-B (BE/ANT-B20/17 and BE/ANT-B13/17) replicated very well in HEp-2 and A549 cell lines and quite well in BEAS-2B. Overall, differences in infection kinetics were observed within the different clinical isolates.

### Thermal Stability

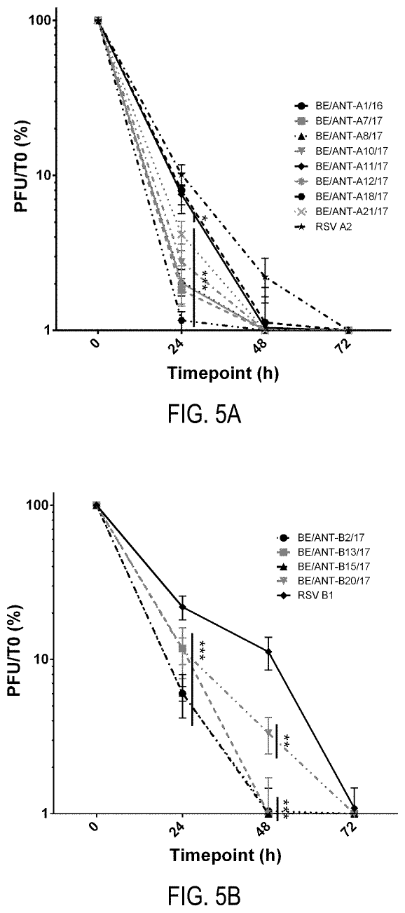

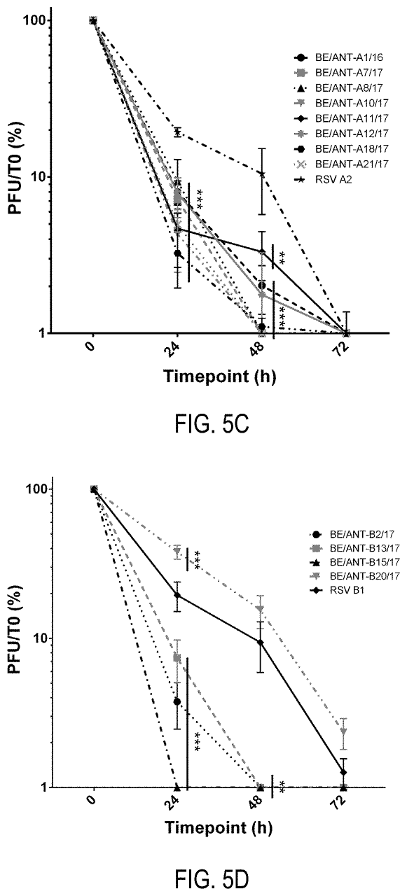

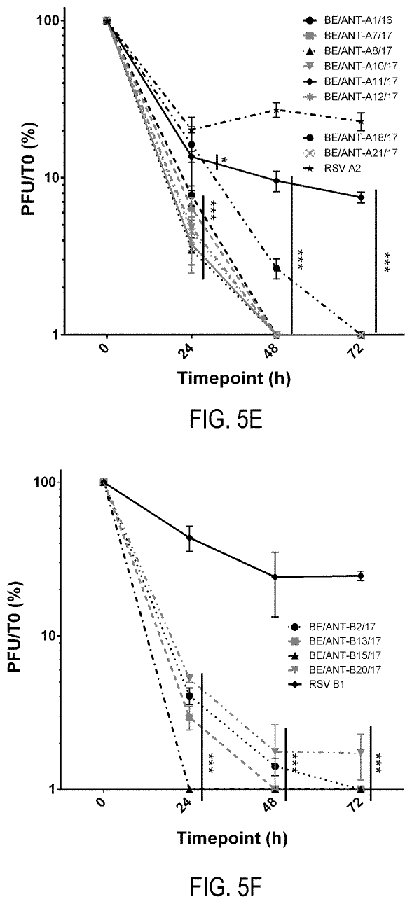

Differences in the F protein are shown to be involved in thermal stability of viral particles [7]. Aliquots of each virus containing 1×10⁵ PFU/ml were incubated at three different temperatures: 37° C. (in vitro incubator temperature and core body temperature) (FIG. 5A and B), 32° C. (upper airway temperature) (FIG. 5C and D) and 4° C. (storage temperature) (FIG. 5E and F) for 24 h, 48 h and 72 h. Aliquots were snap frozen in liquid nitrogen and used for conventional plaque assay to quantify infectious virus. For all RSV-A isolates and RSV A2, higher temperatures were associated with a faster decay of infectious virus. Curiously, BE/ANT-A11/17 conserved higher PFU at 4° C. than other RSV-A isolates although at the other temperatures there was no difference. Also BE/ANT-A18/17 was preserved slightly better at 4° C., however at 72 h no viable virus was detected. RSV-B isolate BE/ANT-B20/17 retained higher titers for the duration of the experiment compared to other RSV-B isolates but its overall stability was less than the reference RSV B1. The only exception is at 32° C., where its viral titers remained higher than RSV B1. Isolate BE/ANT-B15/17 seems to decay especially fast at any other temperature than 37° C.

### Cell to Cell Fusion

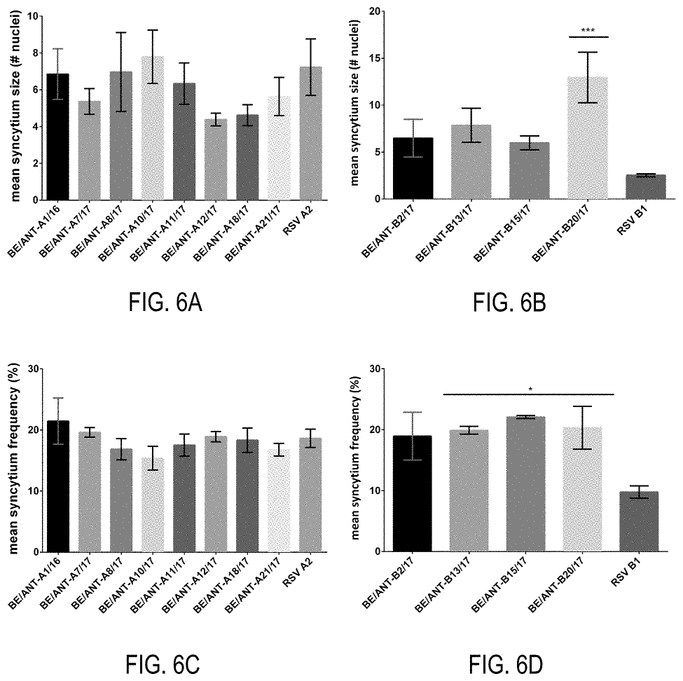

Syncytia formation has long been considered a typical characteristic of RSV infection in immortal cell lines, and it has been used as a measure of activity of the fusion protein [8]. HEp-2 cells were infected with an MOI of 0.05 and incubated for 48 h with an overlay of DMEM¹⁰ containing 0.6% avicel to allow spreading of the infection to neighboring cells only. Afterwards, cells were fixed, fluorescently stained and analyzed with fluorescence microscopy. Mean syncytium size was determined, (FIG. 6A and 6B) as well as mean syncytium frequency (FIG. 6C and 6D) by counting the number of nuclei belonging to syncytia relative to the total number of nuclei of infected cells. Mean syncytium size of all RSV-A clinical isolates (FIG. 6A) lies between four and seven nuclei per cell, with BE/ANT-A1/16, BE/ANT-A8/17 and BE/ANT-A10/17 having the largest syncytia. The smallest syncytia were produced by BE/ANT-A12/17. Mean syncytium frequencies lie between 16% and 21%, with the lowest frequency found for BE/ANT-A10/17, which suggested that it promotes the formation of larger syncytia rather than many small syncytia (FIG. 6C). Clinical isolate BE/ANT-B20/17 formed significantly larger syncytia with a mean size of 13 compared to all clinical isolates (FIG. 6B). Reference strain RSV B1 formed almost no syncytia, with the smallest size and lowest frequency of all viruses tested.

### Plaque Reduction by Palivizumab

Viral neutralization by palivizumab was assessed with a conventional plaque reduction assay. Virus was incubated with a two-fold dilution series of palivizumab for 1 h at 37° C. and then transferred to HEp-2 cells for 2 h at 37° C. to allow infection by non-neutralized virus. Afterwards, the supernatant was replaced by DMEM¹⁰ containing 0.6% avicel and incubated for three days until plaques were visible to the naked eye. Plaques were counted to determine the concentration of palivizumab in which 50% of the virus was neutralized.

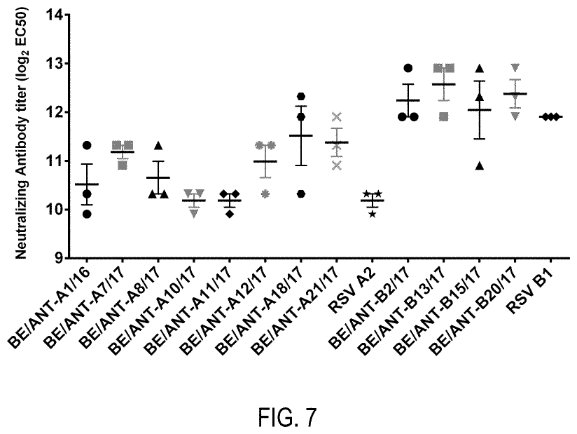

FIG. 7 shows that RSV-A clinical isolates BE/ANT-A7/17 and BE/ANT-A21/17 are 50% neutralized at lower palivizumab concentrations than most of the other clinical isolates and RSV A2, resulting in better neutralization than the other isolates. Remarkably, RSV A2 and RSV B1 neutralization was significantly different, with palivizumab neutralizing the RSV-B strains much better than the RSV-A strains. Overall no significant differences were observed between RSV-B clinical isolates and the reference RSV-B1 for palivizumab neutralization.

### Mucin Expression

RSV infection is hallmarked by an increase of mucus production and impaired mucociliary clearance. As MUCSAC and MUCSB are important players in the secreted airway mucins and MUC1 and MUC4 in the cell-tethered mucins [9], their mRNA expression levels upon RSV infection of A549 cells was tested. mRNA expression levels of the mucins were assessed by infecting A549 cells for 48 h with an MOI of 0.1, followed by qRT-PCR with primers for the different mucin encoding genes. A549 cells were incubated with virus of each isolate for 2 h, after which the inoculum was removed and replaced with DMEM¹⁰. Cells were incubated for 48 h, collected for lysis followed by an RNA extraction and qRT-PCR.

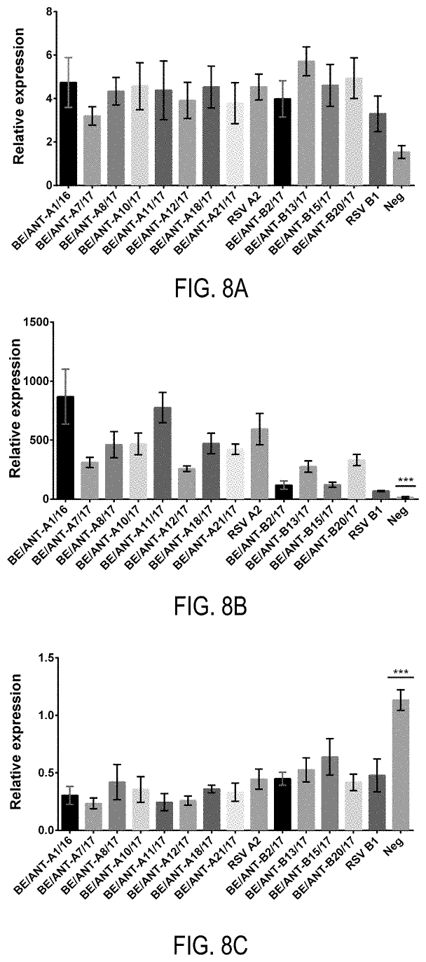

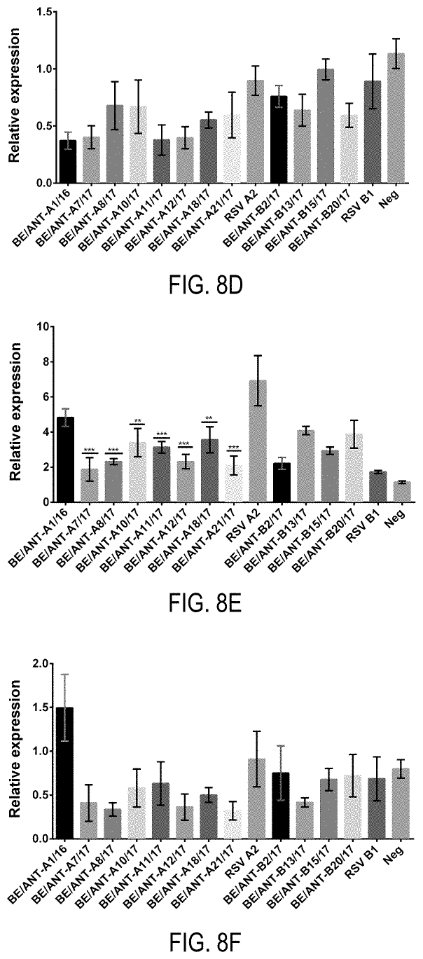

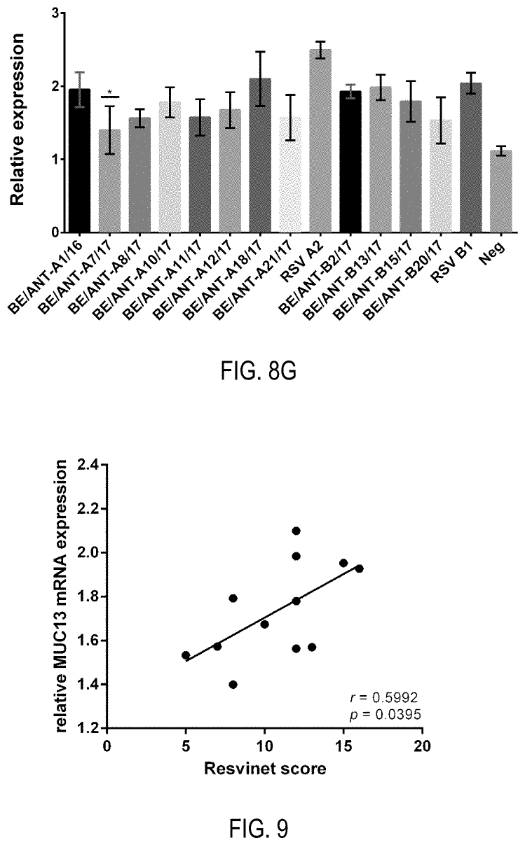

For all clinical isolates and controls, the relative expression of cell-tethered MUC1 (FIG. 8A) is increased compared to the non-infected control. No significant differences can however be observed between RSV isolates and controls.

Expression profiles of the cell-tethered MUC4 show a considerable relative increase compared to the negative control (FIG. 8B). Infection of BE/ANT-A1/16 and BE/ANT-Al 1/17 resulted in the highest relative increases of MUC4 mRNA among all the RSV-A clinical isolates, whereas BE/ANT-A7/17 and BE/ANT-Al2/17 resulted in the lowest increase. For the RSV-B clinical isolates, significantly lower increases are observed when compared to the RSV-A clinical isolates, but an increase is still observed. Infection of isolates BE/ANT-B13/17 and BE/ANT-B20/17 resulted in the highest increase of MUC4 mRNA expression among the RSV-B isolates.

MUC5AC is mainly produced in the epithelial goblet cells, and was previously reported to slightly decrease in A549 cells under the influence of an RSV-infection after 48 h [10]. Here, expression of MUC5AC is significantly reduced upon infection with all clinical isolate infections and reference strains, however no significant differences can be observed between the clinical isolates (FIG. 8C).

MUC5B is produced by surface secretory cells throughout the airways and submucosal glands. Our results show that MUC5B expression is downregulated as a result of RSV infection, with strongest downregulation of RSV-A clinical isolates BE/ANT-A1/16, BE/ANT-A7/17, BE/ANT-A11/17 and BE/ANT-Al2/17. Overall downregulation of MUC5B by the RSV-B clinical isolates is limited, with almost none in infections with BE/ANT-B15/17 (FIG. 8D).

MUC2 expression in A549 cells is overall increased for all RSV infections in comparison to the negative control (FIG. 8E). The expression in RSV-A clinical isolates is significantly different from the RSV A2 prototype strains in BE/ANT-A7/17, BE/ANT-A8/17, BE/ANT-A10/17, BE/ANT-A11/17, BE/ANT-A12/17, BE/ANT-A18/17 and BE/ANT-A21/17. No significant differences can be observed between the RSV-B clinical isolates and the RSV B1 prototype strain.

No significant differences in relative expression of MUC6 can be observed between the clinical isolates and their corresponding prototype strains (FIG. 8F). Expression MUC6 as a result of RSV-A clinical isolates results in a relative decrease compared to the prototype strain RSV A2 and the negative control, except for BE/ANT-A1/16.

Relative expression of MUC13 is generally increased for all clinical isolates and prototype strains compared to the negative control (FIG. 8G). Clinical isolates BE/ANT-A7/17 is significantly decreased compared to prototype strain RSV A2, whereas for all other clinical isolates, no significant differences can be observed compared to the corresponding prototype strains.

### Correlation of Mucin Expression with Clinical Symptoms

mRNA expression levels of the mucins were assessed by infecting A549 cells for 48 h with an MOI of 0.1, followed by qRT-PCR with primers for the different mucin encoding genes. A549 cells were incubated with virus of each isolate for 2 h, after which the inoculum was removed and replaced with DMEM¹⁰. Cells were incubated for 48 h, collected for lysis followed by an RNA extraction and qRT-PCR.

Pearson's correlation was used to determine the relationship between MUC13 mRNA expression and the “Resvinet score”. Said Resvinet score (Justicia-Grande et al., Plos One, 2016) is a clinical scale based on seven parameters (feeding intolerance, medical intervention, respiratory difficulty, respiratory frequency, apnoea, general condition, fever) that were assigned different values (from 0 to 3) for a total of 20 points. The correlation coefficient r was 0.5992 with a p value of 0.0395 (FIG. 9), indicating a positive and linear correlation between the two variables. This analysis thus indicates that the relative mRNA expression of MUC13 in respiratory epithelial cells is positively correlated with RSV disease severity, represented by the Resvinet score.

### F Protein Sequence Analysis

As the F protein regulates the most important function of viral entry, the fusion event, differences in its protein sequence are important to map as well. We sequenced the F-gene of each clinical isolate and translated the coding sequences to their corresponding in-frame protein sequence by aligning them to the corresponding RSV A2 and RSV B1 reference strain. All F proteins of the RSV-A clinical isolates differ from the RSV A2 strains in 12 amino acids, three in the signal peptide, three in the F2 subunit, one residue in the fusion peptide, three in the F1 subunit and one in the HRB and transmembrane domain respectively (data not shown). Between the clinical isolates, several differences can be observed, resulting in all unique F protein sequences.

Compared to the RSV A2 sequences, there are two additional potential N-glycosylation consensus sites present in certain RSV-A clinical isolates compared to RSV A2. In the p27 at residue 122, the substitution of A to T in clinical isolates BE/ANT-A1/17, BE/ANT-A8/17, BE/ANT-A10/17, BE/ANT-A11/17, BE/ANT-A12/17 and BE/ANT-A18/17 results in the consensus sequence N-X-T/S indicating a potential N-glycosylation site, which has been previously seen in other clinical isolates and the RSV Long strain. The remaining two clinical isolates BE/ANT-A7/17 and BE/ANT-A21/17 contain a mutation at residue 120 from an N to a S, effectively removing one N-glycosylation site. Two clinical isolates, BE/ANT-A10/17 and BE/ANT-A12/17 have an additional substitution of an I residue to an N at residue 195, forming a new N-glycosylation consensus sequence, which has never been described before.

### REFERENCES

1. Justicia-Grande A J, Pardo-Seco J, Cebey-Lopez M, Vilanova-Trillo L, Gomez-Carballa A, Rivero-Calle I, et al. Development and validation of a new clinical scale for infants with acute respiratory infection: The resvinet scale. PLoS One. 2016; 11:1-15.

2. Van der Gucht W, Leemans A, De Schryver M, Heykers A, Caljon G, Maes L, et al. Respiratory syncytial virus (RSV) entry is inhibited by serine protease inhibitor AEBSF when present during an early stage of infection. Virol. J. [Internet]. Virology Journal; 2017; 14:157. Available from: www.ncbi.nlm.nih.gov/pubmed/28818113.

3. Schepens B, Sedeyn K, Vande Ginste L, De Baets S, Schotsaert M, Roose K, et al. Protection and mechanism of action of a novel human respiratory syncytial virus vaccine candidate based on the extracellular domain of small hydrophobic protein. EMBO Mol. Med. [Internet]. 2014; 6:1436-54. Available from: embomolmed.embopress.org/cgi/doi/10.15252/emmm.201404005.

4. Houspie L, Lemey P, Keyaerts E, Reijmen E, Vergote V, Vankeerberghen A, et al. Circulation of HRSV in Belgium: From Multiple Genotype Circulation to Prolonged Circulation of Predominant Genotypes. Leung FCC, editor. PLoS One [Internet]. 2013; 8:e60416. Available from: www.ncbi.nlm.nih.gov/pubmed/23577109.

5. Leemans A, Boeren M, Van der Gucht W, Pintelon I, Roose K, Schepens B, et al. Removal of the N-Glycosylation Sequon at Position N116 Located in p27 of the Respiratory Syncytial Virus Fusion Protein Elicits Enhanced Antibody Responses after DNA Immunization. Viruses [Internet]. 2018;10:426. Available from: www.mdpi.com/1999-4915/10/8/426.

6. Tapia L I, Shaw C A, Aideyan L O, Jewell A M, Dawson B C, Haq T R, et al. Gene Sequence Variability of the Three Surface Proteins of Human Respiratory Syncytial Virus (HRSV) in Texas. Varga S M, editor. PLoS One [Internet]. 2014; 9:e90786. Available from: dx.plos.org/10.1371/journal.pone.0090786.

7. Stobart C C, Rostad C A, Ke Z, Dillard R S, Hampton C M, Strauss J D, et al. A live RSV vaccine with engineered thermostability is immunogenic in cotton rats despite high attenuation. Nat. Commun. [Internet]. 2016; 7:13916. Available from: www.nature.com/articles/ncomms13916.

8. Gagliardi T B, Criado M F, Proenca-Modena J L, Saranzo A M, Iwamoto M A, de Paula F E, et al. Syncytia Induction by Clinical Isolates of Human Respiratory Syncytial Virus A. Intervirology [Internet]. 2017; 60:56-60. Available from: www.karger.com/Article/FullText/480014.

9. Ridley C, Thornton D J. Mucins: the frontline defence of the lung. Biochem. Soc. Trans. [Internet]. 2018; 46:1099-106. Available from: www.biochemsoctrans.org/cgi/doi/10.1042/BST20170402.

10. Bañs-Lara M D R, Piao B, Guerrero-Plata A. Differential Mucin Expression by Respiratory Syncytial Virus and Human Metapneumovirus Infection in Human Epithelial Cells. Mediators Inflamm. [Internet]. 2015; 2015:1-7. Available from: www.hindawi.com/journals/mi/2015/347292/.

1-14. (canceled)

15. A method of determining the severity of an RSV infection in a subject, the method comprising:
obtaining a biological sample from the subject;
isolating RSV from the biological sample;
exposing respiratory epithelial cells to the RSV isolate, wherein the respiratory epithelial cells have been cultured in vitro;
detecting an expression level of one or more mucin genes in the respiratory epithelial cells using a technique selected from the group consisting of: polymerase chain reaction, real-time polymerase chain reaction, reverse transcriptase polymerase chain reaction, hybridization, probe hybridization, quantitative gene expression arrays, western blotting, enzyme-linked immunosorbent assay, immune-chromatography, Luminex assays, CyTOF, and immunofluorescence assays; and
diagnosing the subject with a more severe RSV infection when the one or more mucin genes have an elevated expression level compared to an expression level of a control sample.

16. The method of claim 15, wherein diagnosing the subject with the more severe RSV infection further comprises correlating a greater Resvinet score of the subject with the one or more mucin genes having an elevated expression level.

17. The method of claim 15, wherein the one or more mucin genes are selected from the group consisting of MUC1, MUC2, MUC4, MUC5AC, MUC5B, MUC6 and MUC13.

18. The method of claim 15, wherein the mucin gene is MUC13.

19. The method of claim 15, wherein the biological sample is mucus, sputum, nasopharyngeal aspirate, bronchoalveolar aspirate or a bronchoalveolar tissue biopsy of the subject.

20. The method of claim 15, wherein the respiratory epithelial cells are derived from an epithelial cell line.

21. The method of claim 20, wherein the respiratory epithelial cells are derived from the A549 cell line.

22. The method of claim 15, wherein the respiratory epithelial cells are nasal epithelial cells, pharyngeal epithelial cells, bronchial epithelial cells, or lung epithelial cells.

23. The method of claim 15, further comprising treating a subject with a therapy selected from the group consisting of antiviral agents, RSV-specific antibodies, antibody-like molecules, agents that modulate mucin expression, agents that modulate mucin production, mucus regulators, mucolytics, MARCKS blockade, heat shock protein-70 inhibitors, soluble NSF attachment protein receptors cleavage, Munc inhibitors, P2Y2 agonists and antagonists, and macrolide antibiotics.

24. The method of claim 15, wherein the expression level of the one or more mucin genes is detected using real-time polymerase chain reaction.

25. The method of claim 15, wherein the RSV infection is an acute RSV infection.

26. The method of claim 15, wherein the RSV infection is a chronic RSV infection.

27. The method of claim 15, wherein the RSV infection is pharyngitis, croup, bronchiolitis, or pneumonia.

28. The method of claim 15, wherein the subject is a human subject.

29. The method of claim 28, wherein the human subject is a child with an age less than 12 years.


SEQUENCE LISTING (18 sequences)

SEQ ID NO: 1 - DNA, 26 bp, Artificial Sequence
  RSVQA1 primer
gctcttagcaaagtcaagttgaatga

SEQ ID NO: 2 - DNA, 21 bp, Artificial Sequence
  RSVQA2 primer
tgctccgttggatggtgtaat

SEQ ID NO: 3 - DNA, 33 bp, Artificial Sequence
  RSVQA probe
acactcaacaaagatcaacttctgtcatccagc

SEQ ID NO: 4 - DNA, 26 bp, Artificial Sequence
  RSVQB1 primer
gatggctcttagcaaagtcaagttaa

SEQ ID NO: 5 - DNA, 30 bp, Artificial Sequence
  RSVQB2 primer
tgtcaatattatctcctgtactacgttgaa

SEQ ID NO: 6 - DNA, 35 bp, Artificial Sequence
  RSVQB probe
tgatacattaaataaggatcagctgctgtcatcca

SEQ ID NO: 7 - DNA, 20 bp, Artificial Sequence
  G267FW fw primer
atgcaacaagccagatcaag

SEQ ID NO: 8 - DNA, 23 bp, Artificial Sequence
  F164RV rv primer
gttatcacactggtataccaacc

SEQ ID NO: 9 - DNA, 24 bp, Artificial Sequence
  BGF Fw primer
gcagccataatattcatcatctct

SEQ ID NO: 10 - DNA, 22 bp, Artificial Sequence
  BGR rv primer
tgccccagrtttaatttcgttc

SEQ ID NO: 11 - DNA, 22 bp, Artificial Sequence
  G516R primer
gctgcagggtacaaagttgaac

SEQ ID NO: 12 - DNA, 20 bp, Artificial Sequence
  G284F primer
acctgacccagaatccccag

SEQ ID NO: 13 - DNA, 23 bp, Artificial Sequence
  BGF3 primer
agagacccaaaaacacyagccaa

SEQ ID NO: 14 - DNA, 25 bp, Artificial Sequence
  BGR3 primer
acagggaacgaagttgaacacttca

SEQ ID NO: 15 - DNA, 20 bp, Artificial Sequence
  RSVAB_F1FW primer
ggcaaataacaatggagttg

SEQ ID NO: 16 - DNA, 19 bp, Artificial Sequence
  RSVAB_F1RV
aagaaagatactgatcctg

SEQ ID NO: 17 - DNA, 20 bp, Artificial Sequence
  RSVAB_F2FW
tcaatgatatgcctataaca

SEQ ID NO: 18 - DNA, 22 bp, Artificial Sequence
  RSVAB_F2RV
ggacattacaaataattatgac